(19) United States
(12) Patent Application Publication
(10) Pub. No.: US 2016/0185677 A1
(43) Pub. Date: Jun. 30, 2016

(54) CompRocks Compost Boulders

(71) Applicant: Wendy Kay Turner, Howell, MI (US)

(72) Inventor: Wendy Kay Turner, Howell, MI (US)

(21) Appl. No.: 14/985,384

(22) Filed: Dec. 30, 2015

Related U.S. Application Data
(60) Provisional application No. 62/098,531, filed on Dec. 31, 2014.

(51) Int. Cl. C05F 17/02; C05F 9/02; C12M 1/00

(52) U.S. Cl. CPC C05F 17/0205; C12M 23/18; C05F 9/02; C05F 17/0258

(57) ABSTRACT

CompRocks Composting Boulders are units for composting organic materials which bottomless and rest like rocks and boulders in a landscape or garden. One or more units can be used for the natural creation of compost over a period of a year, depending upon the climate. Compost can be created directly at the site where it is needed simply by moving each unit and mulching the compost into the soil below it. Each unit consists of a hollow body with a lid opening for inputting organic matter and holes for aeration and moisture collection. The smallest units are simply filled annually and staked into the ground next to a particular planting so do not have input lids.

### CROSS-REFERENCE TO RELATED APPLICATION

Patent application Ser. No. 62/098,531. Filing Date 12/31/2014. Provisional Application for US Patent Confirmation No. 5796.

### STATEMENT REGARDING FEDERALLY SPONSORED RESEARCH OR DEVELOPMENT

Not Applicable

### REFERENCE TO DEQUENCE LISTING, A TBLE, OR A COMPUTER PROGRAM LISTING COMPACT DISC APPENDIX (IF APPLICABLE)

Not Applicable

### BACKGROUND OF THE INVENTION

Residential composting is a long established process wherein humus is created from organic matter, generally food and yard waste. The creation of humus averages 12 months to complete and varies depending upon temperature as well as oxygen and moisture availability. Numerous devices and techniques have been developed to expedite the process. These products are designed to speed the growth of humus creating bacteria and include encasing the organic matter in dark plastic containers to maintain warmer temperatures and prevent the loss of moisture, aerating the matter to improve oxygenation and rotating the matter to encourage rapid bacterial growth.

The past inventions are either unsightly or look artificial in their environment. Further, they require the user to remove the completed product, humus, from the unit and transport it to the location where it will be applied to the soil.

The present invention accomplishes the goal of eliminating unsightly, open compost piles and the use of compost bins tht look artificial in their environment. It further offers the ability to create compost at the located is it desired, avoiding the need to transport the finished product.

The US Patent Classification System uses the following class/subclass numbers and descriptions for composting devices: U.S.P.C. System 435/290.1 Composting Apparatus. 435/298.1 Apparatus for the treatment of organic waste material. 220/913 Receptacles, Ventilated Container. 220/908 Receptacles, Trash Container. 47/20.1+ Plant Husbandry, Covers, Shades and Screens. 47/62+ Plant Husbandry, Nutrient Supply. 47/65.5 Plant Husbandry, Pots & Receptacles.

The following are two patented composting devices:

| | | |
|---|---|---|
| U.S. Pat. No. 6,921,661 | July 2005 | Nagy et al |
| U.S. Pat. No. 8,940,529 | January 2015 | Morrison |

### BRIEF SUMMARY OF THE INVENTION

CompRocks Compost Boulders are lightweight, bottomless units for composting organic material. They are shaped like individual rocks and boulders and create compost at a desired location. The units consist of hollow bodies with openings for inputting organic matter and holes for ventilation and moisture control. Each unit sits directly on the soil.

CompRocks Compost Boulders are made of molded plastic and include stake(s) for affixing into the ground to provide secure positioning. They vary in size to accommodate the needs of their user and their environment. The holes for ventilation and moisture control vary with each boulder design from small holes, slits and imbedded screens. A hinged, pivot or swing lid opening blends into each boulder design to maintain its natural appearance. The smallest units can be manufactured without input openings as they are simply filled once, stuck into the ground where desired and left until the composting process is completed. Preserving the natural appearance of the individual units is critical.

### BRIEF DESCRIPTION OF THE DRAWINGS

FIG. 1. FIG. 1 shows a side view of an Individual CompRock Compost Boulder.

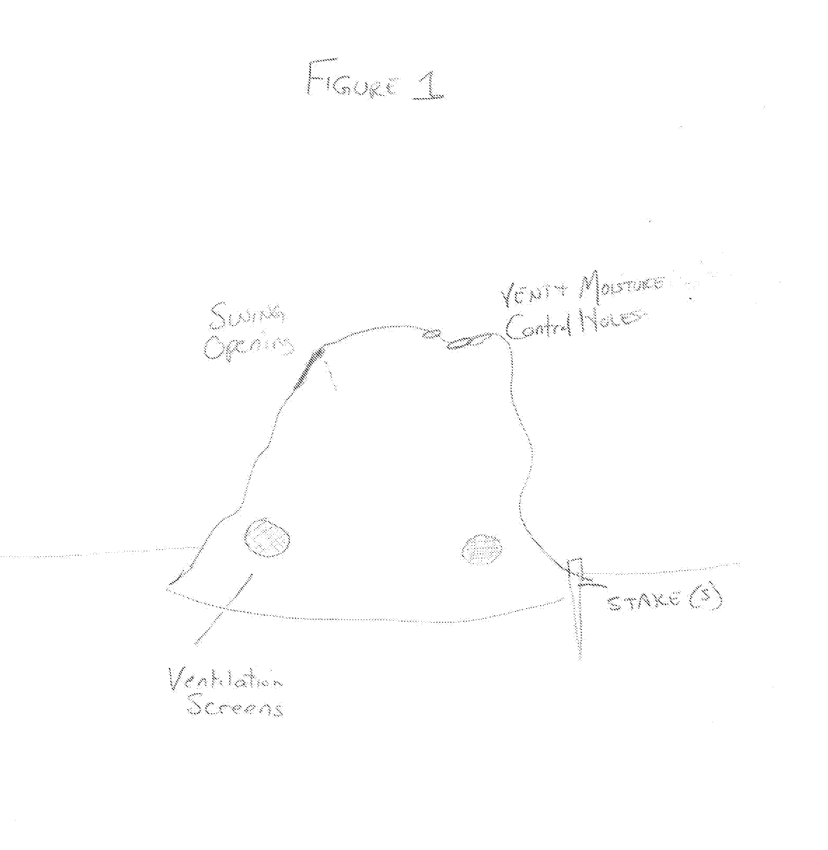

FIG. 2. FIG. 2 illustrates Side/Cut view of a CompRock Compost Boulder with open soil access for earthworms and fungi.

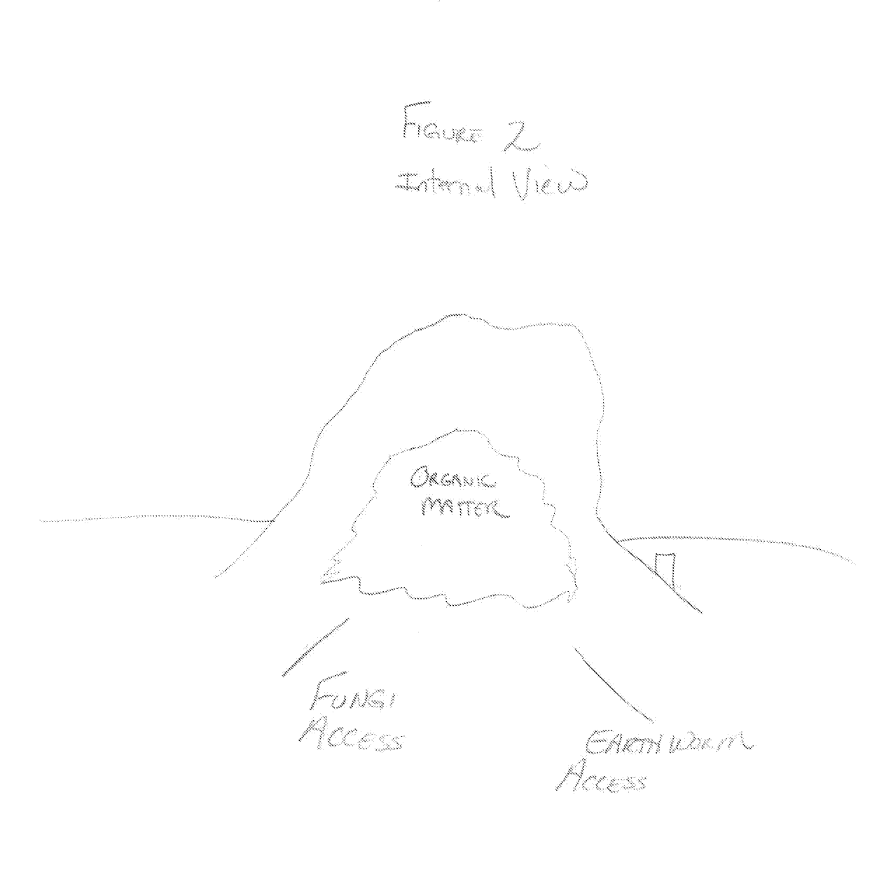

FIG. 3. FIG. 3 presents a side, landscape view of small CompRocks inserted into the soil directly next to individual plantings. These small unit do no have input lids.

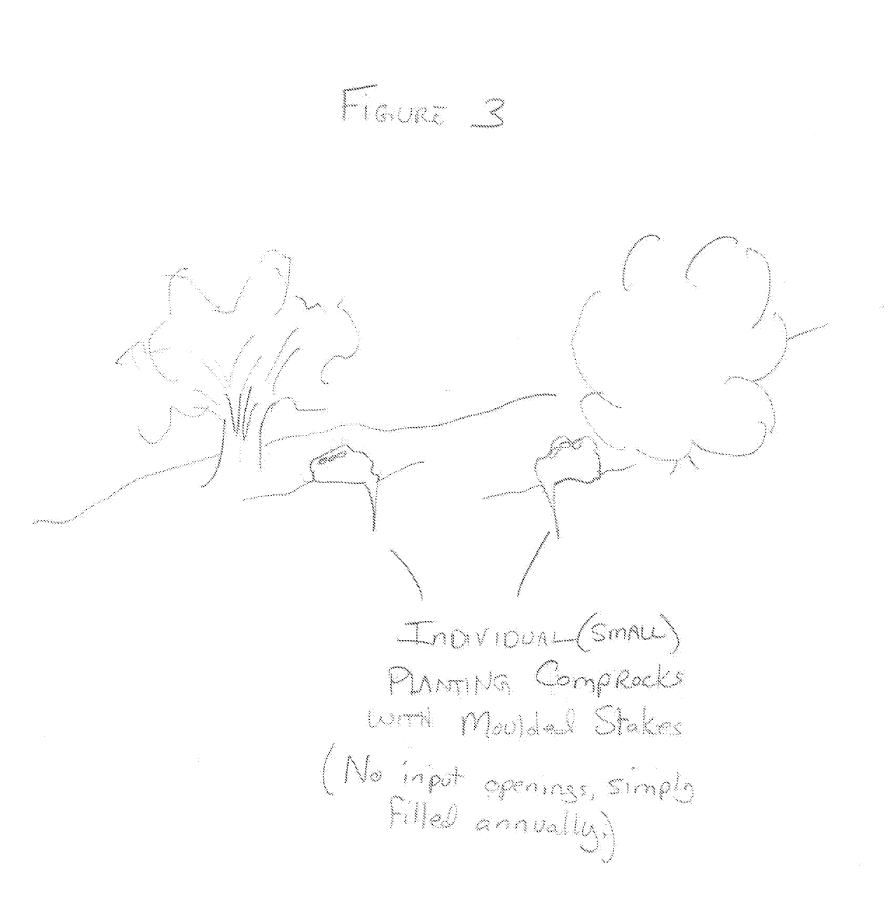

FIG. 4. FIG. 4 presents a top view of a CompRock with the optional pivot lid for inputting organic materials.

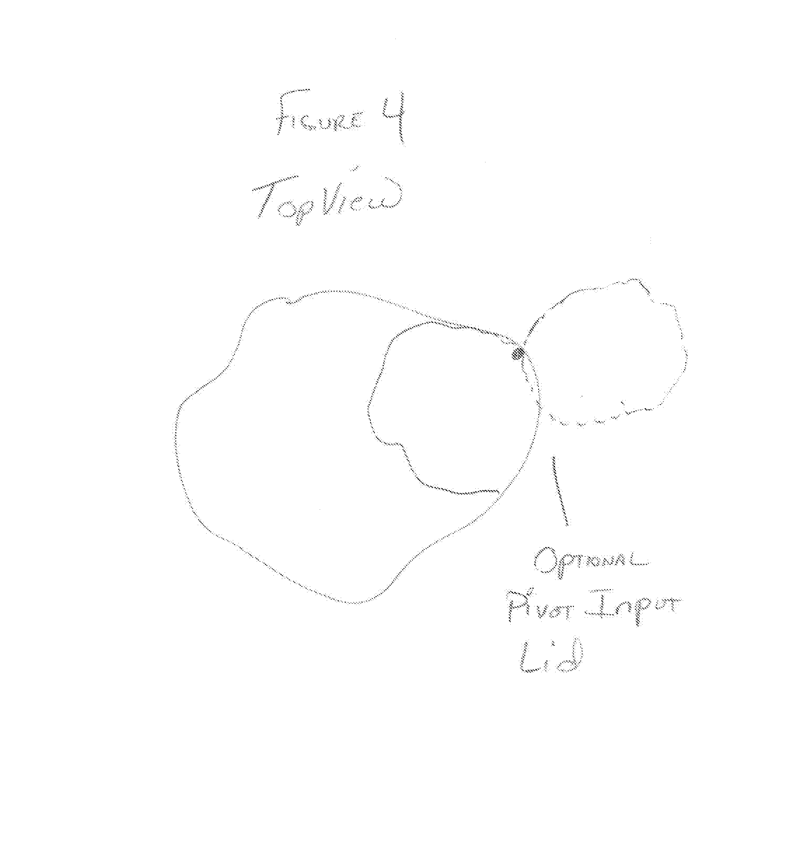

### DETAILED DESCRIPTION OF THE INVENTION

This invention relates to an improved system of composting household biodegradable materials. Specifically, this invention uses the natural features of rocks and boulders that rest directly on the soil to simplify and encourage composting. The system reduces household waste and associated transportation costs as well as the use of artificial fertilizers.

Composting is a long established process wherein humus is created from organic matter, generally food and yard waste. Historically, organic waste was deposited and collected in piles where microorganisms naturally created compost over a period of a year or more, depending upon the climate. Both air and moisture were naturally available in these open piles. More recently, compost bins have been created wherein the organic material is added to a manufactured container with venting holes. The compost bins offer the benefit of an enclosed area which expedites the creation of compost by protecting the microorganisms which create it. Like the compost piles however, many compost bins are unattractive objects that are hidden in a landscape or placed in distant locations to avoid being seen. The compost created in a compost pile and inside of a bin must then be moved to the area where it is to be used. These combined inconveniences deter composting for many households. Recently some compost bins have been created to look more architecturally attractive but they fail to look like natural features in a landscape.

CompRocks Compost Boulders are units for composting organic material that are shaped like rocks and boulders and can be used to create compost at desired locations. Each individual unit consists of a hollow body with a top opening for inputting organic matter and holes for ventilation. In addition to aeration, small vent openings designed into the top of each unit provide for the collection of water, generally rain, as available. The units have open bottoms and rest directly on the soil. When sufficient compost has been created in that location the entire unit can be simply moved to a new location.

The open bottom of each unit provides the same benefit of an open compost pile by allowing access by earthworms and innumerable members of the fungi kingdom. These elements aid in the creation of a richer, more fertile humus. The finished compost provides a free, organic fertilizer to its user while reducing the amount of organic matter in the commercial household waste stream.

CompRocks Compost Boulders are made of heavy duty plastic and include stake(s) for affixing into the ground to provide secure positioning. They vary in size to accommodate the needs of their user and their environment. The holes for aeration vary with each boulder design from small holes, to larger slits, to imbedded screens. The lid opening blends into each boulder design to maintain its natural appearance.

The individual units are naturally attractive rocks and boulder shapes that encourage composting because of their easy access and location. They blend easily and beautifully into a natural environment and can even be arranged to enhance a landscaping. The smallest units are simply filled and staked into the ground next to a particular planting. They are filled annually and provide natural, free, organic fertilizer.

Depending upon the climate, CompRocks Compost Boulders will take approximately 1 year to create the desired compost and a series of larger, seasonal boulders can be used to simplify the process. One boulder, filled in spring, is allowed to develop while the summer, fall and winter boulders are filled in their respective seasons. The following year, the spring boulder can be lifted and moved to a new location and its compost contents mulched into the ground beneath it. The seasonal rotational system can be expanded to provide for smaller or larger increments as needed or desired, ie monthly boulders, biannual boulders, etc.

The invention accomplishes the goal of eliminating unsightly, open compost piles and obvious compost structures. While artificial rocks and boulders made from plastics exist to cover unsightly, stationary landscape objects, they don't provide the ventilation, moisture collection and continuous top access necessary for composting.

The instant invention possesses numerous options for modification to its design, size and detailed appearance and all such variations are believed to remain within the scope of this invention.

1. Composting devices for composting household organic material consisting of boulder and rock shaped, hollow, bottomless units which are affixed directly to the ground, and, depending upon the size of said unit, filled annually or periodically, after which time the entire unit is easily pulled up and moved to a new location, leaving the created compost at the site where it is to be used, avoiding the need for transportation of the end product.

2. The composting device of claim 1 includes the benefits of open-air compost piles because they sit directly on the soil, allowing for an improved compost product due to the access of earthworms and innumerable members of the fungi kingdom.

3. The composting devices of claim 1 include the benefit of appearing as naturally attractive rocks and boulder that do not need to be hidden or placed outside of normal landscaping and therefore encourage composting because of their easy access and central location. They blend easily and beautifully into a natural environment and can even be arranged to enhance a landscaping design.

4. Composting devices which are designed in a variety of sizes to accommodate the needs and desires of the individual user, enabling a unit so small that it can be used to fertilize an individual planting or so large that is can be filled annually with household organic material, each unit being designed with aeration and moisture control holes or embedded screens which virtually eliminate maintenance time and effort by the user, the larger units including input doors for periodic input of organic materials.

5. The composting devices of claim 2, apply a landscape concept of natural features to improve soil health in a customizable, aesthetically appealing means which can be used to the degree individual taste, allowing users to gradually become accustomed to composting and learning the benefits of this art at their own pace, removing the “pass or fail” methodology of traditional composting apparatus.
